(19) United States
(12) Patent Application Publication
(10) Pub. No.: US 2005/0059608 A1
(43) Pub. Date: Mar. 17, 2005

(54) Pharmaceutical compounds

(76) Inventors: Tomi Jarvinen, Kuopio (FI); Jukka Leppanen, Kuopio (FI); Juhani Huuskonen, Jyvaskyla (FI); Jouko Savolainen, Kuopio (FI); Jukka Gynther, Kuopio (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/495,919

(22) PCT Filed: Nov. 18, 2002
(86) PCT No.: PCT/FI02/00915

(30) Foreign Application Priority Data
Nov. 19, 2001 (FI) 20012242

(51) Int. Cl. A61K 38/04; C07K 5/04

(52) U.S. Cl. 514/19; 562/450; 560/41

(57) ABSTRACT

Compounds of formula (I), wherein E, G, T, R_{d}, Rₑ, and R_{f} are as defined in the disclosure, release levodopa and a COMT inhibitor so that they can be used for the treatment of diseases or conditions, wherein levodopa and inhibition of COMT are indicated to be useful.

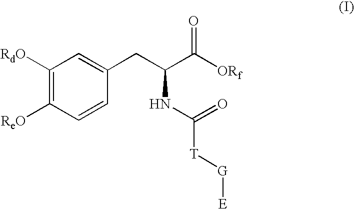

### FIELD OF THE INVENTION

The present invention relates to codrugs of unprotected or suitably protected levodopa and a catechol O-methyltransferase (COMT) inhibitor, or pharmaceutically acceptable salts or esters thereof. The invention further relates to pharmaceutical compositions thereof.

### BRIEF DESCRIPTION OF THE PRIOR ART

The prodrug approach is commonly used to improve physicochemical, biopharmaceutical, and drug delivery properties of therapeutic agents. Ideally, an inactive pro-moiety is attached by covalent bonding to the parent molecule, and the resulting prodrug is converted to the parent drug in the body before it exhibits its pharmacological effect. Many diseases are treated by a combination of therapeutic agents that are co-administered in separate dosage forms.

However, there are potential advantages, e.g. improved delivery properties and targeting drugs to specific sites of action, in giving the co-administered agents as a single chemical entity. In codrugs, at least two synergistic drugs are linked together and designed to release the parent drug at the desired site of action.

Levodopa (3,4-dihydroxyphenyl-L-alanine) is a precursor to dopamine, which is deficient in the brains of patients suffering from Parkinson's disease (PD). Conventional PD treatment consists of levodopa combined with an amino acid decarboxylase (MDC) inhibitor, such as carbidopa. During treatment, COMT remains the main enzyme for metabolizing levodopa. Entacapone [(E)2-cyano-N,N-diethyl-3-(3,4-dihydroxy-5-nitrophenyl)propenamide] is a new, potent inhibitor of COMT. Entacapone is currently used as a clinical adjunct to levodopa therapy in PD treatment. The administration of entacapone, together with levodopa and an MDC inhibitor, leads to increased bioavailability of levodopa and its prolonged duration of action. However, even after combination therapy of entacapone and levodopa, the bioavailability of levodopa is low, i.e. 5-10% [Männistö et al. Pharmacol Toxicol., 66 (1990) 317]. In addition, the bioavailability of entacapone after oral administration is also low, i.e. 29-46% [Keranen et al. Eur. J. Clin. Pharmacol., 46 (1994) 151].

The codrug approach can be considered to be a productive way for combining the therapeutic effects of levodopa and a COMT inhibitor. An effective codrug is stable against chemical hydrolysis, but releases the parent drugs e.g. by enzymatic hydrolysis under physiological conditions.

### SUMMARY OF THE INVENTION

The object of the present invention is to provide compounds that release levodopa and a COMT inhibitor.

The invention also provides compounds for the treatment of diseases or conditions, wherein levodopa and inhibition of COMT are indicated to be useful, as well as a use thereof for the manufacture of a medicament to be used as a precursor for levodopa and a COMT inhibitor. Furthermore, pharmaceutical compositions containing the present compounds are provided.

### DETAILED DESCRIPTION OF THE INVENTION

Levodopa can be linked to the COMT inhibitor via a spacer. Preferably, the COMT inhibitor is a derivative of a catechol compound. Suitable catechol COMT inhibitors for the use of the invention are disclosed e.g. in the following publications: GB 2 200 109 A; U.S. Pat. No. 6,150,412; EP 237 929 B1; and EP 1 010 688 A1.

The present invention thus provides compounds of general formula I, 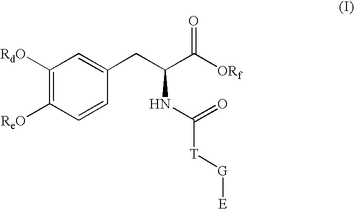
wherein E is a COMT inhibitor moiety; G is —(CO)ₐ—, wherein a is 0 or 1; T is —(CH₂)_{b}—, wherein b is depending on a
if a is 0, then b is 0
if a is 1, then b is 2 or3
R_{d} and Rₑ independently are hydrogen or groups hydrolyzable under physiological conditions, and signify optionally substituted lower alkanoyl or aroyl, lower alkanoylamino, optionally substituted lower alkyl or arylsulphonyl or optionally substituted lower alkylcarbamoyl, or taken together signify a lower alkylidene or cycloalkylidene group; R_{f} is hydrogen or a group hydrolyzable under physiological conditions, and signifies optionally substituted lower alkanoyl or aroyl, lower alkylamino or lower dialkylamino or lower alkanoylamino, optionally substituted lower alkyl or arylsulphonyl or optionally substituted lower alkylcarbamoyl, or pharmaceutically acceptable esters or salts thereof. Preferably, R_{f} is hydrogen or alkyl, e.g. alkyl. Further preferably, R_{d} and Rₑ independently are hydrogen or optionally substituted alkanoyl or aroyl. Compounds, wherein E is a derivative of a catechol compound, are preferred. In the definitions of R_{d}, Rₑ, and R_{f}, the term “lower” denotes residues with a maximum of 8, preferentially a maximum of 4 carbon atoms. The term “alkyl” taken alone or in combination with terms such as “alkanoyl, alkylidene, cycloalkylidene, alkylamino” denotes straight or branched chain saturated or partially unsaturated hydrocarbon residues. The term “aryl” in combination with terms such as “aroyl” denotes a carbocyclic aromatic group, preferably mono- or bicyclic groups. The term “optionally substituted” in connection with various residues refers to halogen substituents, such as fluorine, chlorine, bromine, iodine or trifluoromethyl groups, alkyloxy, or aryl substituents. The “optionally substituted” groups may contain 1 to 3, preferably 1 or 2, most preferably 1 of said substituents.

Compounds of formula I provide adequate stability against chemical hydrolysis at acidic pH, which is a desirable property considering the conditions in the stomach and small intestine, and, additionally, show appropriate biodegradability.

As a subgroup of the compounds of formula 1, the invention provides compounds, wherein E is a catechol COMT inhibitor as disclosed in GB 2 200 109 A, i.e. E is a moiety of formula Ia, 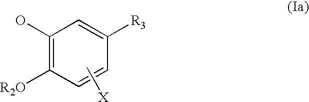
wherein R₂ is hydrogen, optionally substituted acyl or aroyl, lower alkylsulfonyl or alkylcarbamoyl, X comprises an electronegative substituent such as halogen, nitro, cyano, lower alkylsulfonyl, sulfonamido, aldehyde, carboxyl or trifluoromethyl; R₃ is hydrogen, halogen, substituted alkyl, hydroxyalkyl, amino, nitro, cyano, trifluoromethyl, lower alkylsulfonyl, sulfonamido, aldehyde, alkyl carbonyl, aralkylidene carbonyl or carboxyl or a group selected from
—CH═CR₄R₅ and —CH₂CHR₄R₅, wherein R₄ is hydrogen, alkyl, amino, cyano, carboxyl or acyl; and R₅ is hydrogen, amino, cyano, carboxyl, alkoxycarbonyl, carboxy alkenyl, nitro, acyl, hydroxyalkyl, carboxyalkyl or an optionally substituted carboxamido, carbamoyl or aroyl or heteroaroyl, or R₄ and R₅ together form a five to seven membered substituted cycloalkanone ring;
—(CO)ₙ(CH₂)ₘ—COR, wherein n is 0 or 1 and m is 0 or 1-7 and R is hydroxy, alkyl, carboxyalkyl, optionally substituted alkene, alkoxy or optionally substituted amino;
—CONR₈R₉, wherein R₈ and R₉ independently are hydrogen or one of the following optionally substituted groups; alkyl, alkenyl, alkynyl, cycloalkyl, aralkyl, or together form an optionally substituted piperidyl group; and —NH—CO—R₁₀, wherein R₁₀ is a substituted alkyl group. Preferably, R₂ is hydrogen. Further preferably, X is at ortho position to R₂O—. In the definitions of R, R₂, R₃, R₄, R₅, R₆, R₇, R₈, R₉, and R₁₀, the term “alkyl” by itself or as part of another group includes both straight and branched chain radicals of up to 18 carbon atoms, preferably 1 to 8 carbon atoms, most preferably 1 to 4 carbon atoms. The term “lower alkyl” by itself or as part of another group includes both straight and branched chain radicals of 1 to 7, preferably 1 to 4, most preferably 1 or 2 carbon atoms. The terms “alkenyl” and “alkynyl” designate a hydrocarbon residue as defined above with respect to the term “alkyl” including at least one carbon to carbon double bond and carbon to carbon triple bond, respectively. The alkenyl and alkynyl residues may contain up to 12, preferably 1 to 8, most preferably 1 to 4 carbon atoms. The term “acyl” by itself or as part of another group refers to an alkylcarbonyl or alkenylcarbonyl group. The term “aroyl” by itself or as part of another group refers to an arylcarbonyl group, the aryl group being a mono- or bicyclic group containing from 6 to 10 carbon atoms in the ring portion. Specific examples for aryl groups are phenyl, naphthyl, and the like. The term “lower alkylidene” refers to a chain containing from 2 to 8, preferably 2 to 4 carbon atoms. The term “alkoxy” by itself or as part of another group includes an alkyl residue linked to an oxygen atom. The term “cycloalkyl” includes saturated cyclic hydrocarbon groups containing 3 to 8, preferably 5 to 7 carbon atoms. The term “aralkyl” refers to alkyl groups having an aryl substituent. A specific example is the benzyl group. The term “halogen” as refers to chlorine, bromine, fluorine or iodine, chlorine and bromine being preferred. The term “optionally substituted” in connection with various residues refers to halogen substituents, such as fluorine, chlorine, bromine, iodine or trifluoromethyl groups, alkyloxy, aryl, alkyl-aryl, halogen-aryl, cycloalkyl, alkylcycloalkyl, hydroxy, alkylamino, alkanoylamino, arylcarbonylamino, nitro, cyano, thiol, or alkylthio substituents. The “optionally substituted” groups may contain 1 to 3, preferably 1 or 2, most preferably 1 of said substituents. The term “heteroaroyl” refers to mono- or bicyclic groups containing 1 to 3, preferably 1 or 2 heteroatoms N and/or O and/or S.

As a further subgroup of the compounds of formula I, the invention provides compounds, wherein E is a catechol COMT inhibitor as disclosed in U.S. Pat. No. 6,150,412, i.e. E is a moiety of formula Ib, 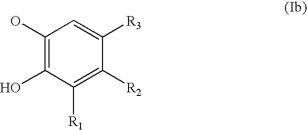
wherein R₁ is an electronegative substituent, preferably nitro, cyano, formyl or carboxy; R₂ is -A-R₄, wherein A is branched or straight chain (C₁₋₉)alkylene; R₄ is carboxy, 5-tetrazolyl, R₅ or CO—R₅, wherein R₅ is phenyl or (C₃₋₇)cycloalkyl which is substituted by at least one carboxy or 5-tetrazolyl; R₃ is an electronegative substituent, preferably nitro, cyano, halogen, formyl, carboxy, (C₁₋₅)alkylcarbonyl, arylcarbonyl or SO₂R₆, wherein R₆ is branched or straight chain (C₁₋₅)alkyl, arylalkyl, aryl or NR₇R₈, wherein R₇ and R₈ are independently hydrogen or branched or straight chain (C₁₋₅)alkyl, or together form a (C₃₋₆)ring, the term “aryl” meaning phenyl or naphthyl.

As a further subgroup of the compounds of formula I, the invention provides compounds, wherein E is a catechol COMT inhibitor as disclosed in EP 237 929 B1, i.e. E is a moiety of formula Ic, 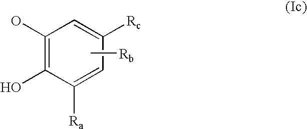
wherein Rₐ is nitro or cyano; R_{b} is hydrogen or halogen, R_{c} is halogen, nitro, cyano or a group -(A)ₙ-(Q)ₘ-R¹ or -(A)ₙ-Q-R², A is vinylene optionally substituted by lower alkyl, n is 0 or 1, m is 0 or 1, R is —COR^{3,} an aromatic carbocyclic group or an aromatic or partially unsaturated heterocyclic group attached via a carbon atom, R² is hydrogen or an optionally substituted, saturated or partially unsaturated lower hydrocarbon residue, R³ is hydroxy, amino, an optionally substituted, saturated or partially unsaturated lower hydrocarbon residue attached via an oxygen atom or an imino or lower alkylimino group or a saturated, N-containing heterocyclic group attached via a ring nitrogen atom, Q is the group —CO— or >C═N-(Z)ₚR⁴, Z is an oxygen atom or an imino group, p is 0 or 1 and R⁴ is hydrogen or a saturated or partially unsaturated, lower hydrocarbon residue which is optionally substituted and which is optionally attached via a carbonyl group. In the definitions of Rₐ, R_{b}, and R_{c}, the term “lower” denotes residues and compounds with a maximum of 7, preferably a maximum of 4, carbon atoms. The term “alkyl”, taken alone or in combinations, such as “alkyl group”, “alkoxy”, “alkylthio”, and “alkylimino”, denotes straight chain or branched, saturated hydrocarbon residues, for example, such as methyl, ethyl, propyl, isopropyl, n-butyl, s-butyl, i-butyl, t-butyl and the like. The term “saturated or partially unsaturated lower hydrocarbon residue” denotes open chain and cyclic groups and combinations thereof. Examples of saturated and partially unsaturated lower hydrocarbon residues are: lower alkyl groups such as those defined above: lower alkenyl groups, for example, 2-propenyl, 2-butenyl, 3-butenyl, and 2-methyl-2-propenyl; C₃₋₇ cycloalkyl and C₈₋₁₀ bicycloalkyl groups optionally substituted by lower alkyl groups, for example, cyclopropyl, cyclopentyl, 2-methylcyclopentyl, cyclohexyl, and 3-methylcyclohexyl; lower cycloalkenyl groups optionally substituted by lower alkyl groups, for example, 3-cyclopentenyl, 1-methyl-3-cyclopentenyl, and 3-cyclohexenyl; lower alkyl or alkenyl groups substituted by lower cycloalkyl or cycloalkenyl groups, for example, cyclopropylmethyl, cyclopropylethyl, cyclopentylmethyl, cyclohexylmethyl, 2-cyclohexenylmethyl, and 3-cyclopropyl-2-propenyl. The lower alkenyl groups preferably contain 2-4 carbon atoms; the cycloalkyl and cycloalkenyl groups preferably contain 3-6 carbon atoms. The following come into consideration as substituents for the above lower hydrocarbon residues: hydroxy, cyano, nitro, halogen, amino, lower alkylamino, di(lower alkyl)amino, lower alkoxy, lower alkoxycarbonyl, aryl, arylaminocarbonyl, arylcarbonyl, arylcarbonylamino, lower alkanoyloxy, lower alkanoyl, carbamoyl, mono- or di(lower alkyl)carbamoyl, lower alkylenedioxy, trifluoromethyl, carboxy, lower alkanoylamino, lower alkoxycarbonylamino, and lower alkylthio. The saturated or partially unsaturated lower hydrocarbon residues are preferably unsubstituted or mono- or disubstituted. The term “aryl” denotes carbocyclic aromatic groups, preferably mono- or bicyclic groups. Especially preferred carbocyclic aromatic groups are phenyl and naphthyl, especially phenyl. These groups are optionally substituted by halogen trifluoromethyl, nitro, amino, mono- or di(lower alkyl)amino, lower alkyl, lower alkoxy, lower alkylthio, lower alkanoyl, lower alkoxycarbonyl, carboxy, hydroxy, cyano, lower alkanoyloxy, carbamoyl, mono- or di(lower alkyl)carbamoyl, lower alkylenedioxy, lower alkanoylamino or lower alkoxycarbonylamino. The carbocyclic aromatic groups are preferably unsubstituted or mono- or disubstituted. The term “aromatic or partially unsaturated heterocyclic group” preferably denotes a mono-, di- or tricyclic, aromatic or partially unsaturated heterocyclic group with up to five heteroatoms from the group consisting of nitrogen, sulfur, and oxygen. The heterocyclic groups preferably contain 1-4 nitrogen atoms and/or an oxygen or sulfur atom. They are preferably mono- or bicyclic. The heteroatoms are preferably distributed on one or two rings, whereby nitrogen atoms can simultaneously also be components of two rings. The heterocyclic groups are preferably aromatic. They can be substituted and are preferably mono, di- or trisubstituted. As substituents there come into consideration halogen, trifluoromethyl, nitro, carboxy, amino, arylamino, lower alkyl, lower alkoxy, hydroxy, lower alkoxycarbonyl, lower alkanoyl, lower alkanoyloxy, oxo, lower alkylenedioxy, mercapto, lower alkylthio, lower alkylamino, di(lower alkyl)amino, C₃₋₇ cycloalkylamino, C₈₋₁₀ bicycloalkylamino, lower alkanoylamino, lower alkoxycarbonylamino, carbamoyl, mono- or di(lower alkyl)carbamoyl, cyano, aryl, aryl(lower alkyl), aryl(lower alkyl)amino, heteroaryl, heteroaryl(lower alkyl), heteroarylamino, and C₃₋₇ cycloalkyl. The monocyclic heterocyclic groups are preferably five or six membered and contain a maximum of 4 heteroatoms. The bicyclic heterocyclic groups are preferably eight to ten membered, with the individual rings being preferably five or six membered. The following are to be mentioned as examples of such heterocyclic groups: pyridyl, pyrazinyl, triazinyl, thiadiazinyl, thiazolyl, oxazolyl, oxadiazolyl, pyrazolyl, tetrazolyl, imidazolyl, thienyl, quinolinyl, isoquinolinyl, dihydroisoquinolinyl, benzoxazinyl, quinoxalinyl, benzopyranyl, benzimidazolyl, indolyl, imidazothiazolyl, imidazothiadiazolyl, imidazopyridyl, benzothiazinyl, benzoquinoxalinyl, and imidazobenzothiazolyl. The term “heteroaryl” denotes aromatic heterocyclic groups, as defined above. The term “saturated, N-containing heterocyclic group attached via a ring nitrogen atom” preferably denotes a three to seven membered, preferably four to six membered, saturated N-heterocycle which, in addition to the said nitrogen atom, can contain an oxygen, sulfur or nitrogen atom as a second heteroatom. These saturated N-heterocycles can be mono- or disubstituted by lower alkyl, hydroxy, lower alkoxy, lower alkanoyloxy, lower hydroxyalkyl, lower alkoxyalkyl, lower alkanoyloxyalkyl, lower alkoxycarbonyl, lower alkanoyl, carbamoyl, mono- or di(lower alkyl)carbamoyl, oxo and/or lower alkylenedioxy. The following are to be mentioned as examples of such N-containing heterocyclic groups: 4-morpholinyl, 1-pyrrolidinyl, and 1-azetidinyl.

As a further subgroup of the compounds of formula I, the invention provides compounds, wherein E is a catechol COMT inhibitor as disclosed in EP 1 010 688 A1, i.e. E is a moiety of formula Id, 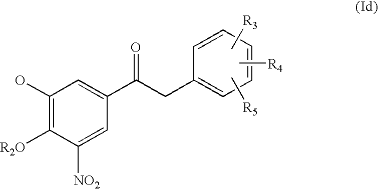
wherein R₂ is hydrogen or a group hydrolyzable under physiological conditions, and signifies optionally substituted lower alkanoyl or aroyl, optionally substituted lower alkyl or arylsulphonyl or optionally substituted lower alkylcarbamoyl; R₃, R₄, and R₅ are the same or different and signify hydrogen, optionally substituted saturated or partially unsaturated lower hydrocarbon residue, hydroxy, optionally substituted lower alkoxy or aryloxy group, optionally substituted aryl, optionally substituted alkanoyl or aroyl group, lower alkanoylamino group, lower dialkanoylamino group, carboxyl, optionally substituted lower alkyloxycarbonyl or aryloxycarbonyl group, optionally substituted carbamoyl, halogen, nitro, amino, lower alkylamino or lower dialkylamino or cyano group, or taken together signify aliphatic or heteroaliphatic rings or aromatic or heteroaromatic rings. Preferably, R₂ is hydrogen. In the definitions of R₂, R₃, R₄, and R₅, the term “lower” denotes residues with a maximum of 8, preferentially a maximum of 4 carbon atoms. The term “alkyl” taken alone or in combination with terms such as “alkanoyl, alkyloxycarbonyl, alkylamino” denotes straight or branched chain saturated hydrocarbon residues. The term halogen denotes fluorine, chlorine, bromine, and iodine. The term “aryl” denotes a carbocyclic aromatic group, preferably mono- or bicyclic groups.

Preferably, the compound is (S)-2-{5-[(E)-2-cyano-2-(diethylcarbamoyl)vinyl]-2-hydroxy-3-nitrophenoxycarbonylamino}-3-(3,4-dihydroxyphenyl)propionic acid methyl ester or (S)-3-(3,4-dihydroxyphenyl)-2-[2-hydroxy-5-(4-methylbenzoyl)-3-nitrophenoxycarbonylamino]propionic acid methyl ester, or pharmaceutically acceptable esters or salts thereof.

Pharmaceutically acceptable salts and esters of all compounds disclosed above, when applicable, may be prepared by known methods. The pharmaceutically acceptable salts are the usual organic and inorganic salts of the art. Such salts are well known in the literature.

The invention provides compounds for the treatment of disorders or conditions wherein levodopa and inhibition of COMT are indicated to be useful, as well as a use thereof for the manufacture of a medicament to be used as a precursor for levodopa and a COMT inhibitor. Furthermore, pharmaceutical compositions containing the present compounds are provided.

The compounds of the invention can be prepared by a variety of synthetic routes analogously to or according to the methods known in the literature using suitable starting materials.

In general, compounds of formula I can be prepared e.g. analogously to or according to scheme 1, 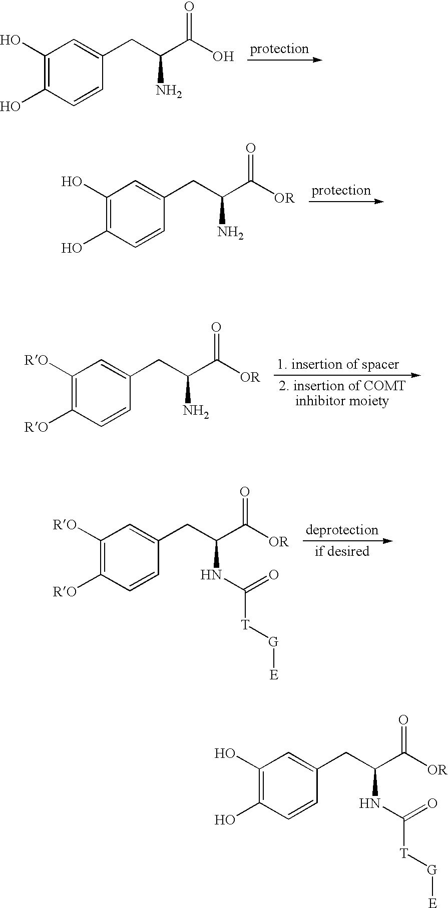
wherein R is e.g. alkyl, R′ is e.g. acyl, and E, G, and T are as defined above.

The carboxylic group of levodopa is protected in a conventional manner, e.g. as an alkyl ester, e.g. as the methyl ester. The hydroxy groups are protected in a conventional manner, e.g. with acyl protecting groups. The desired spacer between the levodopa and COMT inhibitor moieties is accomplished by using appropriate reagents and reactions known in the chemical field, and thereafter the COMT inhibitor moiety can be inserted by known methods. This can be achieved e.g. via an isocyanate or via a dicarboxylic acid monoamide as shown in the specific examples. The protected hydroxy groups can, if desired, be removed in a conventional manner.

The synthetic routes described above are meant to illustrate the preparation of the compounds of the invention and the preparation is by no means limited thereto, i.e. there are also other possible synthetic methods which are within the general knowledge of a person skilled in the art.

The compounds of the invention may be converted, if desired, into their pharmaceutically acceptable salts or esters using methods well known in the art.

The compounds of the invention may be administered enterally, topically or parenterally.

The compounds according to this invention are given to a patient as such or in combination with one or more other active ingredients and/or suitable pharmaceutical excipients. The latter group comprises conventionally used excipients and formulation aids, such as fillers, binders, disintegrating agents, lubricants, solvents, gel forming agents, emulsifiers, stabilizers, colorants and/or preservatives.

The compounds used in this invention are formulated into dosage forms using commonly known pharmaceutical manufacturing methods. The dosage forms can be e.g. tablets, capsules, granules, suppositories, emulsions, suspensions or solutions. Depending on the route of administration and the galenic form, the amount of the active ingredient in a formulation can typically vary between 0.01 and 100% (w/w).

The present invention will be explained in more detail by the following examples. The examples are meant for illustrating purposes only and do not limit the scope of the invention defined in the claims.

### EXAMPLE 1

### (S)-2-{5-[(E)-2-cyano-2-(diethylcarbamoyl)vinyl]-2-hydroxy-3-nitrophenoxycarbonylamino}-3-(3,4-dihydroxyphenyl)propionic acid methyl ester

Levodopa (2 g, 10 mmol) was treated with thionyl chloride (5 ml) in dry methanol (10 ml). The resulting white solid was stirred with trifluoroacetic acid (4 ml) and acetyl chloride (1.5 ml) at room temperature to give (S)-2-amino-3-(3,4-diacetoxyphenyl)propionic acid methyl ester with quantitative yield and high purity. The HCl salt of (S)-2-amino-3-(3,4-diacetoxyphenyl)propionic acid methyl ester (1.5 g, 4.5 mmol) was dissolved in dry ethyl acetate and diphosgene (1.1 ml, 9.0 mmol) was added while stirring at −10° C. under nitrogen atmosphere. (Care must be exercised in the handling of diphosgene due to release of phosgene when heated.) The mixture was allowed to warm to room temperature, then refluxed for 5 h and evaporated to dryness under high vacuum to give (S)-3-(3,4-diacetoxyphenyl)-2-isocyanatopropionic acid methyl ester. The isocyanate product was used immediately in the next reaction without further purification. The product was dissolved in dry acetonitrile (10 ml) with entacapone (553 mg, 1.81 mmol) under nitrogen atmosphere in the absence of light. The mixture was refluxed for 20 h and evaporated to dryness. The product was purified by flash chromatography on silica gel using dichloromethane/methanol (100:1) as an eluent. The acetyl groups were removed by treating with an acetone/3N HCl (20:1) solution for 2 h at 50° C. The resulting clear yellow mixture was evaporated to dryness and purified by preparative HPLC using acetonitrile/water (50:50) as an eluent. Evaporation of solvents yielded (S)-2-{5-[(E)-2-cyano-2-(diethylcarbamoyl)vinyl]-2-hydroxy-3-nitrophenoxycarbonylamino}-3-(3,4dihydroxyphenyl)propionic acid methyl ester as a yellow solid (436 mg, 46%), m.p. (decomposed). ¹H NMR (CDCl₃, TMS) δ: 1.26 (6H, br, CH₂CH₃), 2.95 (1H, q, J=6.1 and 13.7 Hz, CH_{A}CH), 3.11 (1H, q, J=4.7 and 13.7 Hz, CH_{B}CH), 3.50 (4H, br, CH₂CH₃), 3.77 (3H, s, OCH₃), 4.59 (1H, q, J=5.9 and 7.0 Hz, CH₂CH), 6.14 (1H, d, J=7.5 Hz, NH), 6.15 (1H, d, J=8.0 Hz, ArH), 6.66 (1H, s, ArH), 6.72 (1H, d, J=8.0 Hz, ArH), 7.52 (1H, s, CH═C), 7.92 (1H, s, J=1.8 Hz, ArH), 8.32 (1H, s, J=1.8 Hz, ArH). ¹³C NMR (CD₃OD) δ: 12.5, 13.6, 37.0, 41.1, 43.6, 52.7, 55.4, 107.0, 115.5, 116.6, 121.4, 122.9, 124.9, 127.6, 130.0, 134.5, 141.3, 143.3, 143.9, 144.0, 148.1, 151.1, 152.7, 162.9, 171.4. ESI-MS: 543.1 (M+1).

### EXAMPLE 2

### (S)—N-{2-[3,4-bis-(2,2-dimethylpropionyloxy)phenyl]-1-(methoxycarbonyl)ethyl}succinamic acid 5-[(E)-2-cyano-2-(diethylcarbamoyl)vinyl]-2-hydroxy-3-nitrophenyl ester

Levodopa (3.0 g, 15.3 mmol) was mixed with methanol (75 ml) and cooled to 0° C. Thionyl chloride was added during 15 min and the mixture was stirred at room temperature over night. The solvent was evaporated and the oily residue was treated with dry diethyl ether. The formed solid material was filtered and dried under vacuum to give the HCl salt of (S)-2-amino-3-(3,4-dihydroxyphenyl)propionic acid methyl ester. Yield 3.7 g (quant.). The HCl salt of (S)-2-amino-3-(3,4-dihydroxyphenyl)propionic acid methyl ester (1.5 g, 6.07 mmol) was dissolved in trifluoroacetic acid (10 ml). The mixture was stirred and cooled to 0° C. and pivaloyl chloride (1.5 g, 12.4 mmol) was added dropwise during 15 min. The mixture was stirred at room temperature for 2 h. The solvent was evaporated and the residue was dissolved in water. The water solution was neutralized with 5% NaHCO₃ (aq.) solution and extracted four times with dichloromethane. The combined organic layers were dried and evaporated to give (S)-2-amino-3-[3,4-bis-(2,2-dimethylpropionyloxy)phenyl]propionic acid methyl ester. Yield 2.0 g (87%). A solution of (S)-2-amino-3-[3,4-bis-(2,2-dimethylpropionyloxy)phenyl]propionic acid methyl ester (1.2 g, 3.2 mmol), succinic acid anhydride (0.38 g, 3.8 mmol) and 4-(dimethylamino)pyridine (0.47 g, 3.9 mmol) in ethyl acetate (20 ml) was refluxed for 24 h. After cooling, the reaction mixture was washed with 1 M citric acid solution (50 ml). The organic layer was separated and dried over MgSO₄ and evaporated under vacuum. The residue was chromatographed over silica using ethyl acetate as an eluent to give (S)—N-{2-[3,4-bis-(2,2-dimethylpropionyloxy)phenyl]-1-(methoxycarbonyl)ethyl}succinamic acid. Yield 1.3 g (86%). (S)—N-{2-[3,4-bis-(2,2-dimethylpropionyloxy)phenyl]-1-(methoxycarbonyl)ethyl}succinamic acid (1.00 g, 2.08 mmol) and entacapone (0.64 g, 2.10 mmol) were dissolved in ethyl acetate (15 ml). Dicyclohexylcarbodiimide (0.51 g, 2.47 mmol) and 4-(dimethylamino)pyridine (15 mg) were added and stirring was continued for24 h. The insoluble material was filtered and the filtrate was extracted with 5% NaHCO₃ (aq.) solution. The organic layer was separated, dried, and evaporated. The dark red residue was chromatographed over silica using ethyl acetate as an eluent to give (S)—N-{2-[3,4-bis-(2,2-dimethylpropionyloxy)phenyl]-1-(methoxycarbonyl)ethyl}succinamic acid 5-[(E)-2-cyano-2-(diethylcarbamoyl)vinyl]-2-hydroxy-3-nitrophenyl ester as a yellow solid. Yield 0.7 g (44%). ¹H NMR ((CD₃)₂CO, TMS) δ: 1.22 (6H, s (broad), CH₃CH₂), 1.31 (9H, s, (CH₃)₃C), 1.32 (9H, s, (CH₃)₃C), 2.77 (2H, t, CH₂CH₂), 2.90 (2H, t, CH₂CH₂), 3.05 (1H, dd, CH₂CH), 3.15 (1H, dd, CH₂CH), 3.51 (4H, s (broad), NCH₂CH₃), 3.66 (3H, s, CH₃O), 4.83 (1H, q, CH₂CH), 6.50 (1H, q, NH), 7.05 (1H, d, J⁴=1.8 Hz, ArH), 7.08 (1H, d, J³=8.2 Hz, ArH), 7.12 (1H, dd, J⁴=1.8 Hz, J³=8.2 Hz, ArH), 7.61 (1H, s, CH═C), 7.99 (1H, d, ArH), 8.48 (1H, d, ArH). ¹³C NMR ((CD₃)₂CO) δ: 13.39, 27.44, 29.72, 30.86, 37.27, 39.58, 42.77, 52.45, 54.38, 106.19, 116.97, 119.72, 124.07, 125.02, 125.25, 126.69, 127.91, 136.13, 136.35, 137.79, 142.44, 143.32, 143.88, 147.72, 163.97, 171.23, 172.08, 172.78, 176.01, 176.04.

HPLC

The HPLC system used consisted of a Beckman System Gold Programmable Solvent Module 126, Beckman System Gold Detector Module 166 with variable wavelength UV detector (set at 254 nm) and a Beckman System Gold Autosampler 507e. Separations were accomplished on a Purospher RP-18 reverse-phase column, 12.5 cm×4.0 mm i.d., 5 μm (Merck, Darmstadt, Germany). The chromatographic conditions were as follows: injection volume, 50 μl; column temperature, 40° C.; flow rate, gradient/isocratic at 1.0 ml/min. The mobile phase consisted of various proportions of methanol/water mixture (90:10) and a citrate/phosphate buffer pH 2.2.

Hydrolysis in Aqueous Solution

The rate of chemical hydrolysis was determined in aqueous phosphate buffer solution (0.16 M) at pH 7.4, 5.0, and 1.2 at 37° C. An appropriate amount was dissolved in 10 ml of preheated buffer and the solution was placed in a thermostatically controlled water bath at 37° C. At appropriate time intervals, samples were taken and analyzed for the remaining codrug by HPLC. Pseudo-first order half-time (t_{1/2}) for the hydrolysis was calculated from the slope of the linear portion of the plotted logarithm of remaining codrug vs. time.

(S)-2-{5-[(E)-2-cyano-2-(diethylcarbamoyl)vinyl]-2-hydroxy-3-nitrophenoxycarbonylamino}-3-(3,4-dihydroxyphenyl)propionic acid methyl ester: t_{1/2}=12.1 h (pH 1.2); 1.4 h (pH 5.0); 1.1 h (pH 7.4)

Hydrolysis in 10% Rabbit Liver Homogenate

The rabbit liver was homogenized with approximately four equivalent volumes of isotonic phosphate buffer at pH 7.4 using an X-1020 homogenizer (Ystral, Germany). The homogenate was centrifuged for 90 min at 9,000 g and 4° C. with a Biofuge 28 RS centrifuge (Heraeus Instruments, Germany). The supernatant was stored at −80° C. until analysis. An appropriate amount was dissolved in one volume of preheated 20% liver homogenate. The solution was then incubated at 37° C. At appropriate time intervals, samples (300 μl) were withdrawn. Samples were pretreated with 300 μl of methanol to terminate enzymatic activity. After mixing and centrifugation, 400 μl of the supernatant was evaporated to dryness under a stream of air. The residue was dissolved in 400 μl of the mobile phase buffer and analyzed by HPLC.

(S)-2-{5-[(E)-2-cyano-2-(diethylcarbamoyl)vinyl]-2-hydroxy-3-nitrophenoxycarbonylamino}-3-(3,4-dihydroxyphenyl)propionic acid methyl ester: t_{1/2}=7 min (pH 7.4, 37° C.)

1. A compound of general formula I, 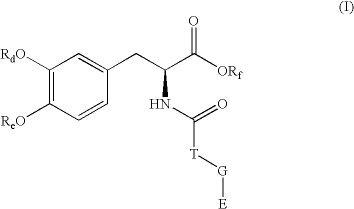
wherein
E is a COMT inhibitor moiety;
G is —(CO)ₐ—, wherein a is 0 or 1;
T is —(CH₂)_{b}—, wherein b is depending on a
if a is 0, then b is 0
if a is 1, then b is 2 or 3;
R_{d} and Rₑ, independently, are hydrogen or a group hydrolyzable under physiological conditions, and signify optionally substituted lower alkanoyl or aroyl, lower alkanoylamino, optionally substituted lower alkyl or arylsulphonyl or optionally substituted lower alkylcarbamoyl, or
R_{d} and Rₑ taken together signify a lower alkylidene or cycloalkylidene group;
R_{f} is hydrogen or a group hydrolyzable under physiological conditions, and signifies optionally substituted lower alkanoyl or aroyl, lower alkylamino or lower dialkylamino or lower alkanoylamino, optionally substituted lower alkyl or arylsulphonyl, or optionally substituted lower alkylcarbamoyl;
or a pharmaceutically acceptable ester or salt thereof.

2. A compound according to claim 1, wherein R_{f} is hydrogen or alkyl.

3. A compound according to claim 1, wherein R_{f} is alkyl.

4. A compound according to claim 1, wherein R_{d} and Rₑ, independently, are hydrogen or optionally substituted alkanoyl or aroyl.

5. A compound according to claim 1, wherein E is a derivative of a catechol compound.

6. A compound according to claim 1, wherein E is a moiety of formula Ia, 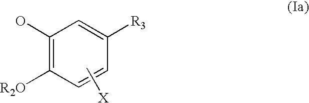
wherein
R₂ is hydrogen, optionally substituted acyl or aroyl, lower alkylsulfonyl or alkylcarbamoyl,
X comprises an electronegative substituent;
R₃ is hydrogen, halogen, substituted alkyl, hydroxyalkyl, amino, nitro, cyano, trifluoromethyl, lower alkylsulfonyl, sulfonamido, aldehyde, alkyl carbonyl, aralkylidene carbonyl or carboxyl or a group selected from
—CH═CR₄R₅ and —CH₂CHR₄R₅, wherein
R₄ is hydrogen, alkyl, amino, cyano, carboxyl or acyl; and
R₅ is hydrogen, amino, cyano, carboxyl, alkoxycarbonyl, carboxy alkenyl, nitro, acyl, hydroxyalkyl, carboxyalkyl or an optionally substituted carboxamido, carbamoyl or aroyl or heteroaroyl, or
R₄ and R₅ together form a five to seven membered substituted cycloalkanone ring;
—(CO)ₙ(CH₂)ₘ—COR, wherein
n is 0 or 1 and
m is 0 or 1-7 and
R is hydroxy, alkyl, carboxyalkyl, optionally substituted alkene, alkoxy or optionally substituted amino;
—CONR₈R₉, wherein
R₈ and R₉, independently, are hydrogen or one of the following optionally substituted groups; alkyl, alkenyl, alkynyl, cycloalkyl, or aralkyl, or
R₈ and R₉ together form an optionally substituted piperidyl group; and
—NH—CO—R₁₀, wherein
R₁₀ is a substituted alkyl group.

7. A compound according to claim 6, wherein R₂ is hydrogen.

8. A compound according to claim 6, wherein X is at an ortho position to R₂O—.

9. A compound according to claim 1, wherein E is a moiety of formula Ib, 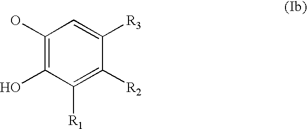
wherein
R₁ is an electronegative substituent;
R₂ is -A-R₄,
wherein
A is branched or straight chain (C₁₋₉)alkylene;
R₄ is carboxy, 5-tetrazolyl, R₅ or CO—R₅,
wherein R₅ is phenyl or (C₃₋₇)cycloalkyl which is substituted by at least one carboxy or 5-tetrazolyl; and
R₃ is an electronegative substituent.

10. A compound according to claim 9, wherein R₁ is nitro, cyano, formyl or carboxy.

11. A compound according to claim 9, wherein
R₃ is nitro, cyano, halogen, formyl, carboxy, (C₁₋₅)alkylcarbonyl, arylcarbonyl or SO₂R₆,
wherein R₆ is a branched or straight chain (C₁₋₅)alkyl, arylalkyl, aryl or NR₇R₈,
wherein R₇ and R₈ are, independently, hydrogen or branched or straight chain (C₁₋₅)alkyl, or together form a (C₃₋₆)ring.

12. A compound according to claim 1, wherein E is a moiety of formula Ic, 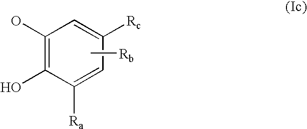
wherein
Rₐ is nitro or cyano;
R_{b} is hydrogen or halogen,
R_{c} is halogen, nitro, cyano or a group -(A)ₙ-(Q)ₘ-R¹ or -(A)ₙ-Q-R²,
wherein
A is vinylene optionally substituted by lower alkyl,
n is 0 or 1,
m is 0 or 1,
R¹ is —COR³, an aromatic carbocyclic group or an aromatic or partially unsaturated heterocyclic group attached via a carbon atom,
R² is hydrogen or an optionally substituted, saturated or partially unsaturated, lower hydrocarbon residue,
R³ is hydroxy, amino, an optionally substituted, saturated or partially unsaturated, lower hydrocarbon residue attached via an oxygen atom or an imino or lower alkylimino group, or a saturated, N-containing, heterocyclic group attached via a ring nitrogen atom,
Q is the group —CO— or >C═N-(Z)ₚ-R⁴,
wherein
Z is an oxygen atom or an imino group,
p is 0 or 1 and
R⁴ is hydrogen or a saturated or partially unsaturated, lower hydrocarbon residue, which is optionally substituted and which is optionally attached via a carbonyl group.

13. A compound according to claim 1, wherein E is a moiety of formula Id, 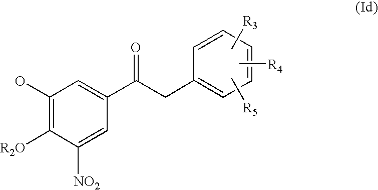
wherein
R₂ is hydrogen or a group hydrolyzable under physiological conditions, and signifies optionally substituted lower alkanoyl or aroyl, optionally substituted lower alkyl or arylsuiphonyl or optionally substituted lower alkylcarbamoyl;
R₃, R₄, and R₅ are the same or different and signify hydrogen, optionally substituted saturated or partially unsaturated lower hydrocarbon residue, hydroxy, an optionally substituted lower alkoxy or aryloxy group, optionally substituted aryl, an optionally substituted alkanoyl or aroyl group, a lower alkanoylamino group, a lower dialkanoylamino group, carboxyl, an optionally substituted lower alkyloxycarbonyl or aryloxycarbonyl group, optionally substituted carbamoyl, halogen, nitro, amino, lower alkylamino or lower dialkylamino or cyano group, or
R₃, R₄, and R₅ taken together signify aliphatic or heteroaliphatic rings or aromatic or heteroaromatic rings.

14. A compound according to claim 13, wherein R₂ is hydrogen.

15. (S)-2-{5-[(E)-2-cyano-2-(diethylcarbamoyl)vinyl]-2-hydroxy-3-nitrophenoxycarbonylamino}-3-(3,4-dihydroxyphenyl)propionic acid methyl ester, or a pharmaceutically acceptable ester or salt thereof.

16. (S)-3-(3,4-dihydroxyphenyl)-2-[2-hydroxy-5-(4-methylbenzoyl)-3-nitrophenoxycarbonylamino]propionic acid methyl ester, or a pharmaceutically acceptable ester or salt thereof.

17-19. (Canceled)

20. A pharmaceutical composition comprising a compound as claimed in claim 1, 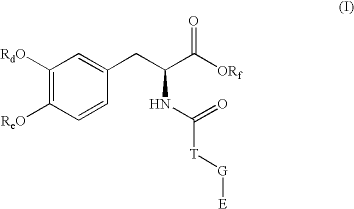
and further comprising a pharmaceutically acceptable excipient.

21. A pharmaceutical composition according to claim 20, wherein the compound is (S)-2-{5-[(E)-2-cyano-2-(diethylcarbamoyl)vinyl]-2-hydroxy-3-nitrophenoxycarbonylamino}-3-(3,4-dihydroxyphenyl)propionic acid methyl ester, or a pharmaceutically acceptable ester or salt thereof.

22. A pharmaceutical composition according to claim 20, wherein the compound is (S)-3-(3,4-dihydroxyphenyl)-2-[2-hydroxy-5-(4-methylbenzoyl)-3-nitrophenoxycarbonylamino]propionic acid methyl ester, or a pharmaceutically acceptable ester or salt thereof.

23. A method for the treatment of diseases or conditions, wherein levodopa and inhibition of COMT are indicated to be useful, said method comprising administering to a mammal in need of such treatment an effective amount of a compound as claimed in claim 1,

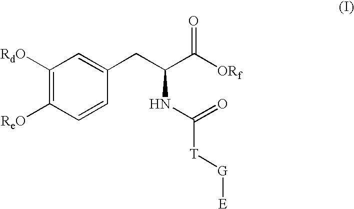

24. A method according to claim 23, wherein the compound is (S)-2-{5-[(E)-2-cyano-2-(diethylcarbamoyl)vinyl]-2-hydroxy-3-nitrophenoxycarbonylamino}-3-(3,4-dihydroxyphenyl)propionic acid methyl ester, or a pharmaceutically acceptable ester or salt thereof.

25. A method according to claim 23, wherein the compound is (S)-3-(3,4-dihydroxyphenyl)-2-[2-hydroxy-5-(4-methylbenzoyl)-3-nitrophenoxycarbonylaminol]propionic acid methyl ester, or a pharmaceutically acceptable ester or salt thereof.

26. A compound according to claim 6, wherein X comprises halogen, nitro, cyano, lower alkylsulfonyl, sulfonamido, aldehyde, carboxyl, or trifluoromethyl.
